(19) United States
(12) Patent Application Publication
(10) Pub. No.: US 2010/0286458 A1
(43) Pub. Date: Nov. 11, 2010

(54) METHOD FOR ISOMERIZING OLEFINS

(75) Inventors: Stefan Iselborn, Frankenthal (DE); Thomas Heidemann, Viernheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/738,034

(22) PCT Filed: Oct. 15, 2008
(86) PCT No.: PCT/EP2008/063859

§ 371 Date: Apr. 14, 2010

(30) Foreign Application Priority Data
Oct. 15, 2007 (EP) 07118502.9

(51) Int. Cl. C07C 5/03; C07C 5/25

(52) U.S. Cl. 585/253; 585/277; 585/324; 585/329; 585/667; 585/670

(57) ABSTRACT

The present invention relates to a process for isomerizing olefins in olefin-comprising hydrocarbon mixtures having from 4 to 20 carbon atoms at temperatures of from 20 to 200° C. and pressures of from 1 to 200 bar in the liquid phase in the presence of a heterogeneous catalyst, wherein a catalyst comprising from 1 to 20% by weight of nickel in oxidic form and from 1 to 20% by weight of at least one element of group VIB on an aluminum oxide support is used.

The present invention relates to a process for isomerizing olefins in olefin-comprising hydrocarbon mixtures having from 4 to 20 carbon atoms, in particular a process for isomerizing 1-butene to 2-butene.

Numerous processes for the double bond isomerization of olefins which are carried out either at temperatures above 250° C. or at lower temperatures in the range from 100 to 250° C. or even below 100° C. both with and without addition of hydrogen are known from the prior art. The temperature level has a critical influence on the isomer composition. Thus, the formation of olefins having internal double bonds occurs preferentially at relatively low temperatures, while mainly 1-olefins are formed at higher temperatures. The equilibrium composition is dependent not only on the temperature but also on the olefin used.

As is known from, inter alia, U.S. Pat. No. 6,156,947, U.S. Pat. No. 5,087,780 and U.S. Pat. No. 4,417,089, the double bond isomerization can be carried out using hydrogen over catalysts comprising noble metals. These processes are often carried out in combination with a diolefin hydrogenation. Alkanes are frequently formed by overhydrogenation as a secondary reaction in the isomerization.

To avoid overhydrogenation during the isomerization, processes for double bond isomerization which can be carried out without addition of hydrogen are therefore also known.

Suitable catalysts for such isomerization reactions are, for example, the alkaline earth metal oxides on aluminum oxide described in EP 0 718 036 A1 and the mixed aluminum oxide/silicon oxide supports doped with the oxides of the alkaline earth metals, boron group metals, lanthanides or elements of the iron group which are known from U.S. Pat. No. 4,814,542 or γ-aluminum oxide treated with alkali metals as is described in JP 51-108691.

Further suitable catalysts are catalysts comprising manganese oxide on aluminum oxide, described in U.S. Pat. No. 4,289,919; catalysts comprising magnesium oxide, alkali metal oxides and zirconium oxide dispersed on an aluminum oxide support, described in EP 0 234 498 A1; and aluminum oxide catalysts additionally comprising sodium oxide and silicon oxide, described in U.S. Pat. No. 4,229,610. However, the catalysts mentioned have the disadvantage that they display a significant isomerization activity only at reaction temperatures above 250° C.

For this reason, superbasic catalysts based on alkali metal (suboxide)/support are used in the isomerization processes described in DE 23 36 138. These can also be used at temperatures below 100° C. A disadvantage here is the high moisture sensitivity of the catalyst.

It was therefore an object of the invention to provide a process for isomerizing olefins, in particular for isomerizing n-butenes, which makes the selective formation of olefins having internal double bonds in high yields at low temperatures possible. Furthermore, the formation of undesirable dienes, e.g. 1,3-butadiene, should be avoided in the isomerization.

This object has been achieved by a process for isomerizing olefins in olefin-comprising hydrocarbon mixtures having from 4 to 20 carbon atoms at temperatures of from 20 to 200° C. and pressures of from 1 to 200 bar in the liquid phase in the presence of a heterogeneous catalyst, wherein a catalyst comprising from 1 to 20% by weight, preferably from 5 to 15% by weight, particularly preferably from 7 to 12% by weight, of nickel in oxidic form and from 1 to 20% by weight, preferably from 2 to 12% by weight, particularly preferably from 3 to 9% by weight, of at least one element of group VIB on an aluminum oxide support is used, where the percentages by weight indicate the amounts of the respective metals based on the total weight of the catalyst used according to the invention.

The designation of the groups of the Periodic Table of the Elements is according to the CAS (chemical abstracts service) nomenclature.

As group VIB element, preference is given to using tungsten or molybdenum or mixtures of tungsten and molybdenum, in each case in oxidic form, with particular preference being given to using tungsten in oxidic form.

In addition, the catalyst can further comprise from 0.1 to 10% by weight, preferably from 0.3 to 5% by weight, particularly preferably from 0.5 to 2% by weight, of one or more elements of group VB in oxidic form, in particular vanadium, and/or from 0.1 to 1% by weight, preferably from 0.1 to 0.8% by weight, particularly preferably from 0.1 to 0.5% by weight, of boron or phosphorus or a mixture of boron and phosphorus, in each case in oxidic form.

The catalyst can additionally comprise from 0.01 to 0.5% by weight, preferably from 0.1 to 0.4% by weight, of sulfur in oxidic form, with the proviso that the ratio of sulfur to nickel is in the range from 0.01 to 0.1 mol/mol, in order to suppress typical secondary reactions such as skeletal isomerization and oligomerization effectively.

The aluminum oxide used as support material for the catalysts used in the process of the invention is preferably gamma-Al₂O₃, theta-Al₂O₃ or eta-Al₂O₃ or mixtures thereof, as are, for example, commercially available from the companies BASF, SASOL, Alcoa, Grace and Rhone-Poulenc. Particular preference is given to aluminum oxide supports which comprise predominantly gamma-Al₂O₃. These aluminum oxides preferably have a water uptake capacity of from 0.2 to 1.5 ml/g of support material, preferably from 0.4 to 1.0 ml/g of support material, and an internal surface area, measured by the BET method, of from 100 to 600 m²/g, preferably from 120 to 450 m²/g, particularly preferably from 150 to 350 m²/g. Further preference is given to such aluminum oxides having a content of less than 0.2% of Na₂O, 0.2% of Fe₂O₃ and/or 0.1% of SO₃.

The active components, additives and/or dopants comprised in the catalyst can be applied to the support by any known method, for example by coating from the gas phase (chemical or physical vapor deposition) or impregnation of the support material with a solution comprising the substances and/or compounds to be deposited.

Impregnation processes for the deposition of active components, additives and/or dopants on a support are known. In general, the support is impregnated with an aqueous or alcoholic solution of salts of the components to be deposited which during the course of the further production of the catalyst are converted into the substances to be deposited, with the volume of the solution being such that the solution is taken up virtually completely by the pore volume of the support (“incipient wetness” method).

Suitable VIB compounds are all compounds of group VIB which can be converted into an oxidic form of the metal on heating in the presence of oxygen or of oxygen-comprising gas mixtures such as air under the calcination conditions. Preference is given to using water-soluble group VIB salts, especially ammonium tungstates, ammonium molybdates, molybdic acids, tungstic adds or heteropolyacids in the H or NH₄ form, as VIB compound.

Suitable nickel compounds are all compounds of nickel which can be converted into an oxidic form of the metal on heating in the presence of oxygen or of oxygen-comprising gas mixtures such as air under the calcination conditions. Preference is given to using water-soluble nickel salts, for example with an organic anion such as formate, oxalate, acetylacetonate or 2-ethylhexanoate and especially hydrated or anhydrous nickel nitrate, as nickel compound.

The substances to be deposited can be deposited individually and/or in partial amounts in a plurality of process steps or together and completely in one process step. Joint deposition in one impregnation step is preferred. The concentration of the salts in the solution is calculated so that after impregnation and conversion of the supported catalyst into the finished catalyst the components to be deposited are present in the desired concentration on the catalyst. The salts are selected so that they leave no residues which interfere in catalyst production or later use of the catalyst.

Water-soluble compounds of sulfur, boron, phosphorus, vanadium and/or niobium which can be converted into an oxidic form of the element on heating in the presence of oxygen or oxygen-comprising gas mixtures such as air under the calcination conditions can optionally be additionally added to the above impregnation solution.

After impregnation, the impregnated support is dried in a customary manner. This is generally effected in a stream of air at a temperature in the range from 60 to 300° C., preferably in the range from 80 to 250° C., particularly preferably in the range from 100 to 200° C., very particularly preferably in the range from 110 to 180° C. Drying is continued until water present in the impregnated catalyst has been given off essentially completely, which is generally the case after a few hours. Usual drying times are in the range from 1 to 30 hours and depend on the drying temperature set, with a higher drying temperature shortening the drying time. Drying can be accelerated further by use of subatmospheric pressure.

In a preferred embodiment of the process of the invention, drying of the impregnated catalyst is carried out with simultaneous movement of the impregnated support material, for example in a rotary tube oven.

In a further preferred embodiment of the process of the invention, the air stream used for drying is passed through the rotary tube in countercurrent.

After drying, the catalyst is produced by calcination in a customary manner. This calcination serves essentially to convert the salts applied by impregnation into the components to be deposited or precursors of such components. In the case of application of metal nitrates by impregnation, the nitrates are decomposed during this calcination essentially into metals and/or metal oxides which remain in the catalyst and nitrous gases which are given off.

The catalytically active oxidic active composition comprising nickel and group VIB element is formed from the nickel compound and the VIB compound during calcination.

The calcination temperature is generally in the range from 200 to 900° C., preferably from 280 to 800° C., particularly preferably from 300 to 600° C. The calcination time is generally in the range from 0.5 to 20 hours, preferably from 0.5 to 10 hours, particularly preferably from 0.5 to 5 hours. Calcination is carried out in a conventional furnace, for example in a rotary tube furnace, in a belt calciner or a box furnace. The calcination can directly follow drying without the impregnated and dried support being cooled in between.

In a particularly preferred embodiment of the process of the invention, drying and calcination of the catalyst are carried out in a combined fashion in a rotary tube furnace.

The catalysts produced in this way are advantageously subjected to conditioning in a dry stream of gas (e.g. dry nitrogen), e.g. at atmospheric pressure and temperatures of from 20 to 500° C., preferably from 100 to 250° C., in order to remove traces of moisture (for instance from the air) from the catalyst before they are used as isomerization catalyst.

A fixed-bed reactor is preferably used for the isomerization process of the invention. It is also possible to use other types of reactor, e.g. a fluidized-bed reactor, a moving-bed reactor, tube reactors or shell-and-tube reactors. The reaction is slightly exothermic. The reaction can be carried out isothermally or adiabatically.

The isomerization is carried out at a temperature at which translocation of the double bond is ensured but skeletal isomerizations and oligomerizations are largely avoided. The reaction temperature is therefore generally in the range from 20 to 200° C., preferably from 20 to 120° C., particularly preferably from 30 to 110° C. The pressure is set so that the olefin stream is in liquid form. It is generally from 1 to 200 bar, preferably from 1 to 100 bar, particularly preferably from 4 to 30 bar.

The WHSV over the catalyst is generally from 0.01 to 20 kg, preferably from 0.05 to 15 kg, particularly preferably from 0.1 to 10 kg, of olefin to be polymerized per kg of catalyst per hour.

Suitable starting materials are olefins or olefin-comprising hydrocarbon mixtures having from 4 to 20 carbon atoms and a high proportion of 1-olefins. In particular, both pure 1-butene and hydrocarbon mixtures having a high proportion of 1-butene, for example C4 fractions as are obtained in steam cracking or FCC or in the dehydrogenation of butane, can be used for the process of the invention. In this sense, high means a content which is higher than the content at thermodynamic equilibrium under the temperatures set in the reaction.

The isomerization process of the invention can be carried out alone or in combination with other chemical processes. Typical examples of combined process steps, which do not, however, restrict the invention, are:
Combination A:
   selective hydrogenation (e.g. of butadiene in a C4-olefin stream from steam crackers) and isomerization according to the invention;
Combination B:
   isomerization according to the invention and olefin metathesis (e.g. reaction of ethylene with 2-butenes to produce propylene);
Combination C:
   isomerization according to the invention and olefin alkylation (e.g. reaction of long-chain linear internal olefins with maleic anhydride to form alkylsuccinic anhydrides);
Combination D:
   isomerization according to the invention and olefin oligomerization (e.g. to prepare alkylate (reaction of i-butene with n/1-butenes)).

Preferred process combinations which comprise the isomerization process of the invention are the combinations A and B.

Olefin metathesis has in recent years become an extremely valuable tool in organic synthesis. On the industrial scale, too, a series of applications have become established, for example the process of Shell AG (SHOP process) for preparing internal olefins and, in particular, the Phillips process for preparing propene by ethenolysis (metathetic cleavage by means of ethene) of 2-butene, with the metathesis step in each case representing an important building block.

However, one important point which has for a long time strongly influenced the development of industrial processes stands in the way of the valuable range of applications of the metathesis reaction: metathesis catalysts deactivate relatively quickly compared to other catalyst systems employed in industry. Owing to the often expensive transition metal catalysts used for their metathesis activity, it is desirable to reduce or avoid the deactivation attributable, for example, to impurities in the feed.

The cause of the deactivation of metathesis catalysts has already been discussed in detail in the literature. Examples are J. Mol. Cat. 1991, 65, pages 39 to 50 (Commereuc et al.), Catalysis today 1999, 51, pages 289 to 299 (J. C. Mol) and J. Mol. Cat. 1991, 65, pages 219 to 235 (J. C. Mol).

In principle, two deactivation routes have been postulated in the literature, namely an intrinsic route which is always present and a deactivation mechanism which is caused by particular impurities in the feed stream. These impurities in the feed stream can have a reversible effect or act as permanent poisons.

In particular, acetylenic compounds, isobutene and 1,3-butadiene are mentioned as deactivating substances in the literature since they tend to form oligomers by cationic mechanisms and these oligomers function as a diffusion barrier. A further important class of deactivating substances which has been mentioned is polar, basic components. This influence is known and is avoided in the prior art by the use of adsorptive guard beds (e.g. molecular sieves) to purify the feed. A detailed examination of the influence of oxygen-comprising compounds on metathesis catalysts may be found in J. A. K. du Plissis, J. Mol. Cat. A: Chemical, 1989, 133, pages 181 to 186. Zeolites or aluminum oxides, in particular, can be used for adsorptive feed purification.

The prior art has described selective hydrogenation as a measure against the 1,3-dienes and acetylenic compounds present in the C4 feed.

Thus, EP 0 742 195 A1 describes a process for converting C4 or C5 fractions into ethers and propylene. Proceeding from C4 fractions, diolefins and acetylenic impurities present are firstly selectively hydrogenated, with the hydrogenation being associated with an isomerization of 1-butene to 2-butene. The yield of 2-butenes should be maximized. The ratio of 2-butene to 1-butene after the hydrogenation is about 9:1. This is followed by etherification of the isoolefins comprised, with the ethers being separated from the C4 fraction. Oxygen-comprising impurities are then separated off. The output stream comprising alkanes and predominantly 2-butene is then reacted with ethylene in the presence of a metathesis catalyst in order to obtain a reaction product mixture comprising propylene as product. The metathesis is carried out in the presence of a catalyst comprising rhenium oxide on a support.

DE-A 198 13 720 relates to a process for preparing propene from a C4 stream. Here, butadiene and isobutene are firstly removed from the C4 stream. Oxygen-comprising impurities are then separated off and a two-stage metathesis of the butenes is carried out. 1-Butene and 2-butene are firstly reacted to form propylene and 2-pentene, and the 2-pentene obtained is then reacted further with added ethylene to form propylene and 1-butene.

DE 100 13 253 A1 describes suitable pretreatments for C4 streams which are used for metathesis. Here, the removal of 1,3-butadiene and acetylenic compounds is achieved by extraction and/or selective hydrogenation. The limit value for the sum of dienes is defined as less than 10 ppm in DE 100 13 253 A1.

Olefin-comprising hydrocarbon mixtures which comprise 1-butene and 2-butene and possibly isobutene are obtained, inter alia, as C4 fraction in various cracking processes such as steam cracking or fluid catalytic cracking. As an alternative, butene mixtures as are obtained in the dehydrogenation of butanes or by dimerization of ethene can be used. Butanes comprised in the C4 fraction behave as inerts. Dienes, alkynes or enynes are removed by customary methods such as extraction or selective hydrogenation before the metathesis step according to the invention.

The butene content of the C4 fraction used in the process is from 1 to 100% by weight, preferably from 50 to 90% by weight. The butene content is based on 1-butene, 2-butene and isobutene.

Preference is given to using a C4 fraction as is obtained in steam cracking or fluid catalytic cracking or in the dehydrogenation of butane.

Selective hydrogenation of crude C4 fraction

Butadienes (1,2- and 1,3-butadiene) and alkynes or alkenynes comprised in the crude C4 fraction from a steam cracker or a refinery are firstly selectively hydrogenated in a generally two-stage process. The C4 stream from a refinery can also be fed directly into the second step of the selective hydrogenation.

The first step of the hydrogenation is preferably carried out over a catalyst comprising from 0.1 to 0.5% by weight of palladium on aluminum oxide as support. The reaction is carried out in the gas/liquid phase in a fixed bed (downflow mode) with circulation of liquid. The hydrogenation is carried out at a temperature in the range from 40 to 80° C. and a pressure of from 10 to 30 bar, a molar ratio of hydrogen to butadiene of from 10 to 50 and an LHSV of up to 15 m³ of fresh feed per m³ of catalyst per hour and a ratio of recycle to fresh feed of from 5 to 20.

The second step of the hydrogenation is preferably carried out over a catalyst comprising from 0.1 to 0.5% by weight of palladium on aluminum oxide as support. The reaction is carried out in the gas/liquid phase in a fixed bed (downflow mode) with circulation of liquid. The hydrogenation is carried out at a temperature in the range from 50 to 90° C. and a pressure of from 10 to 30 bar, a molar ratio of hydrogen to butadiene of from 1.0 to 10 and an LHSV of from 5 to 20 m³ of fresh feed per m³ of catalyst per hour and a ratio of recycle to fresh feed of from 0 to 15.

This hydrogenation generally gives a C4-olefin mixture having a 1,3-butadiene content of from 100 to 500 ppm, preferably from 110 to 400 ppm, particularly preferably from 120 to 300 ppm, and a content of cumulated dienes such as propadiene, 1,2-butadiene, 1,2-pentadiene or 2,3-pentadiene of less than 10 ppm, preferably from 1 to 10 ppm, particularly preferably from 2 to 10 ppm.

The product stream obtained after the selective hydrogenation can then be used directly in the isomerization reaction described at the outset.

Thus, for example, the isomerization of n-butenes is a very important process step in the preparation of propylene from n-butenes and ethylene in the metathesis process because, as mentioned in U.S. Pat. No. 6,743,958, the selectivity to propylene and thus the yield of propylene product and the energy consumption of the process becomes optimal when very little 1-butene but as much 2-butene as possible are present in the starting mixture for the metathesis process.

Depending on the composition of the starting mixture used for the metathesis, a catalytic hydrogenation step and an isomerization step frequently have to be inserted upstream of the metathesis step because, as mentioned above, some constituents of the mixture, e.g. butadienes, have an adverse effect on the metathesis step (e.g. as catalyst poison). According to the prior art, the catalytic hydrogenation step is frequently configured so that the hydrogenation step is, if possible, accompanied by simultaneous hydroisomerization in the hydrogenation reactor.

In US 2006/0235254 A1, it is stated that the isomerization of 1-butene to 2-butene in the hydroisomerization can be increased further by addition of carbon monoxide CO to the starting material or to the hydrogen. In U.S. Pat. No. 6,743,958, it is stated that the isomerization can be increased by addition of sulfur to the catalyst in the hydroisomerization. However, here too, the degree of isomerization still remains limited.

There are also undesirable secondary reactions in the hydrogenation, for example overhydrogenation of the butenes to butane, which leads to a reduction in the product yield. For this reason, the hydrogenation process has to be optimized so that the butadiene is hydrogenated but the butene is not. Experts call such hydrogenation processes selective hydrogenations.

Carrying out these processes is very complicated and/or the processes cannot be used everywhere. For example, the catalysts according to U.S. Pat. No. 6,743,958 have to be prepared separately in a time-consuming complex production process. The use of CO as per US 2006/0235254 A1 often does not come into question because CO is toxic and has to be separated off again in subsequent steps before the products of the process can be used.

If, on the other hand, selective hydrogenation and isomerization are carried out in separate process steps, each individual process step can be optimized so that hydrogenation occurs optimally and, in the other process step, 1-butene is optimally isomerized to 2-butene. To carry out the hydrogenation step, it is possible to choose catalysts which are offered by many manufacturers. These are mostly Pd-, Pt- or Ni-comprising catalysts which are often applied to oxidic supports, preferably aluminum oxide supports. In the isomerization step, it is possible to use the catalyst according to the invention.

The selective hydrogenation step and the isomerization step can occur in separate process steps or separate reactors. However, it is also possible to allow the selective hydrogenation step and the isomerization step to proceed in the same reactor, e.g. by installing the different catalysts above one another in the reactor, with the hydrogenation catalyst preferably being installed at the top and the amount of hydrogen being calculated so that the hydrogen is consumed by reaction with the unsaturated hydrocarbons over the hydrogenation catalyst to a residual concentration of less than 0.5 mol of hydrogen/mol of dienes.

The novel isomerization process of the invention thus allows the ratio of 2-butene to 1-butene at the outlet of the isomerization stage to be increased to values above 10 and even above 20. In addition to these process improvements which are advantageous for the preparation of propylene by means of metathesis, only very small amounts of undesirable by-products such as butadienes or oligomers are formed.

### Olefin Metathesis

The metathesis reaction which can be employed in combination with the isomerization process of the invention can be carried out, for example, as described in WO 00/39058 or DE 100 13 253 A1.

Olefin metathesis (disproportionation) in its simple form is the reversible, metal-catalyzed transalkylidenation of olefins by rupture or reformation of C═C double bonds according to the following equation:

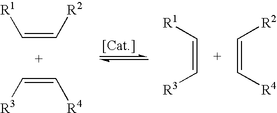

In the specific case of the metathesis of acyclic olefins, a distinction is made between self-metathesis in which an olefin is converted into a mixture of two olefins having a different molar mass (for example: propene→4 ethene+2-butene), and cross-metathesis or cometathesis, namely a reaction of two different olefins (propene+1-butene→ethene+2-pentene). If one of the reactants is ethene, the reaction is generally referred to as an ethenolysis (for example 2-butene+ethene 2-propene).

Suitable metathesis catalysts are in principle homogeneous and heterogeneous transition metal compounds, in particular those of transition groups VI to VIII of the Periodic Table of the Elements, and also homogeneous and heterogeneous catalyst systems comprising these compounds.

For the purposes of the present invention, particular preference is given to metathesis processes starting out from C4 streams having a ratio of 2-butene to 1-butene in the range from 5 to 40.

Thus, DE 199 32 060 A1 describes a process for preparing C₅-/C₆-olefins by reaction of a starting stream comprising 1-butene, 2-butene and isobutene to form a mixture of C₂₋₆-olefins. In particular, propene is obtained from butenes. In addition, hexene and methylpentene are discharged as products. No ethene is introduced in the metathesis. If appropriate, ethene formed in the metathesis is recirculated to the reactor.

The butene content of the C4 fraction used in the process can be in the range from 1 to 100% by weight, preferably from 60 to 90% by weight. The butene content here is based on 1-butene, 2-butene and isobutene.

The C4-olefin mixture used can, if appropriate, be subjected to an appropriate treatment over guard beds of adsorbents, preferably high surface area aluminum oxides or molecular sieves, to free it of interfering impurities before the metathesis reaction.

The metathesis reaction is preferably carried out in the presence of heterogeneous metathesis catalysts which display little if any isomerization activity and are selected from the class of transition metal compounds of metals of group VI.b, VII.b or VIII of the Periodic Table of the Elements applied to inorganic supports.

Preference is given to using rhenium oxide on a support, preferably on γ-aluminum oxide or on Al₂O₃/B₂O₃/SiO₂ mixed supports, as metathesis catalyst.

In particular, Re₂O₇/γ-Al₂O₃ having a rhenium oxide content of from 1 to 20% by weight, preferably from 3 to 15% by weight, particularly preferably from 6 to 12% by weight, is used as catalyst.

The metathesis in the liquid mode of operation is preferably carried out at a temperature of from 0 to 150° C., particularly preferably from 20 to 80° C., and a pressure of from 2 to 200 bar, particularly preferably from 5 to 30 bar.

If the metathesis is carried out in the gas phase, the temperature is preferably from 100 to 450° C., particularly preferably from 200 to 350° C. The pressure in this case is preferably from 1 to 40 bar, particularly preferably from 1 to 30 bar.

The catalysts are used in freshly calcined form and do not require any further activation (e.g. by means of alkylating agents). Deactivated catalyst can be regenerated a number of times by burning off coke residues at temperatures above 400° C. in a stream of air and cooling under an inert gas atmosphere.

In a specific variant of modern-day metathesis processes, the reverse Phillips triolefin process (see J. Mol. Cat. A: Chemical 213 (2004) 39), an isomerization catalyst comprising MgO is used in addition to the actual metathesis catalyst in the metathesis reactor in order to increase the proportion of 2-butene in the recycle stream. However, the proportion of 1-butene in the recycle stream is also quite high here because the thermodynamic equilibrium at the prevailing process temperature above 200° C. restricts the 2-butene to 1-butene ratio which is possible in principle to a value below 10.

The present invention also provides a process for preparing propylene from n-butenes and ethylene by
a) selective hydrogenation of a C4 mixture comprising essentially a mixture of 1- and 2-butenes and butadiene;
b) isomerization of the 1-butene in the selectively hydrogenated C4 mixture at temperatures of from 20 to 200° C. and pressures of from 1 to 200 bar in the liquid phase in the presence of a heterogeneous catalyst;
c) metathesis of ethylene with 2-butene to form propylene in the selectively hydrogenated and isomerized C4 mixture in the presence of a metathesis catalyst, with at least equimolar amounts of ethylene based on the butenes comprised in the C4 mixture being used.

In the isomerization in process step b), it is generally possible to use all known isomerization catalysts. In a preferred embodiment of the abovementioned process, a catalyst comprising from 1 to 20% by weight, preferably from 5 to 15% by weight, particularly preferably from 7 to 12% by weight, of nickel in oxidic form and from 1 to 20% by weight, preferably from 2 to 12% by weight, particularly preferably from 3 to 9% by weight, of at least one element of group VIB on an aluminum oxide support is used in process step b), with the percentages by weight being based on the total weight of the catalyst used according to the invention.

As group VIB element, preference is given to using tungsten or molybdenum or mixtures of tungsten and molybdenum, in each case in oxidic form, with particular preference being given to using tungsten in oxidic form.

In addition, the catalyst can further comprise from 0.1 to 10% by weight, preferably from 0.3 to 5% by weight, particularly preferably from 0.5 to 2% by weight, of one or more elements of group VB in oxidic form, in particular vanadium, and/or from 0.1 to 1% by weight, preferably from 0.1 to 0.8% by weight, particularly preferably from 0.1 to 0.5% by weight, of boron or phosphorus or a mixture of boron and phosphorus, in each case in oxidic form.

The catalyst can additionally comprise from 0.01 to 0.5% by weight, preferably from 0.1 to 0.4% by weight, of sulfur in oxidic form, with the proviso that the ratio of sulfur to nickel is in the range from 0.01 to 0.1 mol/mol, in order to suppress typical secondary reactions such as skeletal isomerization and oligomerization effectively.

In a preferred embodiment of the process of the invention, the C4 mixture used for the selective hydrogenation in process step a) additionally comprises n-butane, isobutane or isobutene or mixtures thereof.

In a further preferred embodiment of the process of the invention, the isomerization in process step b) is, unlike hydroisomerization, carried out without addition of hydrogen.

In metathesis processes customary nowadays, the conversion in the actual metathesis reactor is not complete, which is why the unreacted butenes are very frequently separated off from the propylene product by customary separation methods (e.g. separation columns or the like) and combined with the butenes of the starting material and recirculated to the metathesis reactor. The recycle stream comprises considerable amounts of 1-butene.

In a further preferred embodiment of the abovementioned process of the invention which is derived therefrom,
d) the product stream obtained from the metathesis c) is firstly separated by distillation into a low boiler fraction A comprising C2-C3-olefins and a high boiler fraction B comprising C4-C6-olefins and butanes;
e) the low boiler fraction A obtained from d) is subsequently separated by distillation into an ethylene-comprising fraction and a propylene-comprising fraction, with the ethylene-comprising fraction being recirculated to process step c) and the propylene-comprising fraction being discharged as product, and
f) the high boiler fraction B obtained from d) is combined with the selectively hydrogenated C4 mixture obtained in process step a) and reused in the isomerization b).

In a likewise preferred embodiment of the process of the invention,
d) the product stream obtained from the metathesis c) is firstly separated by distillation into a low boiler fraction A comprising C2-C3-olefins and a high boiler fraction B comprising C4-C6-olefins and butanes;
e) the low boiler fraction A obtained from d) is subsequently separated by distillation into an ethylene-comprising fraction and a propylene-comprising fraction, with the ethylene-comprising fraction being recirculated to process step c) and the propylene-comprising fraction being discharged as product, and
f) the high boiler fraction B obtained from d) is combined with the selectively hydrogenated and isomerized C4 mixture obtained in process step b) and reused in the metathesis c).

The isomerization is carried out at a temperature at which relocation of the double bond is ensured but skeletal isomerizations and oligomerizations are very largely avoided. The reaction temperature is therefore generally from 20 to 200° C., preferably from 20 to 120° C., particularly preferably from 30 to 110° C. The pressure is set so that the olefin stream is in liquid form. It is generally from 1 to 200 bar, preferably from 1 to 100 bar, particularly preferably from 4 to 30 bar.

In a preferred embodiment of the process of the invention, the ratio of 2-butene to 1-butene at the outlet of the isomerization stage b) is in the range from 5 to 40, particularly preferably from 10 to 30.

The selective hydrogenation and metathesis used together with the isomerization according to the invention are known to those skilled in the art from the prior art and the catalysts and reaction conditions used in them have already been described in more detail at the outset.

The present invention also provides a process for preparing propylene from n-butenes and ethylene by
a) selective hydrogenation of a C4 mixture comprising essentially a mixture of 1- and 2-butenes and butadiene;
b) metathesis of ethylene with 2-butene to form propylene in the selectively hydrogenated C4 mixture in the presence of a metathesis catalyst, with at least equimolar amounts of ethylene based on the butenes comprised in the C4 mixture being used,
c) separation of propylene from the reaction mixture;
d) isomerization of the 1-butene in the C4 reaction mixture which has been freed of propylene at temperatures of from 20 to 200° C. and pressures of from 1 to 200 bar in the liquid phase in the presence of a heterogeneous catalyst and
e) combination of the streams from stages a) and d) and reuse in the metathesis stage b),
   wherein a catalyst comprising from 1 to 20% by weight of nickel in oxidic form and from 1 to 20% by weight of at least one element of group VIB on an aluminum oxide support is used in process step d).

In a preferred embodiment of the process of the invention, the catalyst in process step d) further comprises from 0.1 to 10% by weight, preferably from 0.3 to 5% by weight, particularly preferably from 0.5 to 2% by weight, of one or more elements of group VB, in particular vanadium, in oxidic form.

In a further preferred embodiment of the process of the invention, the catalyst in process step d) further comprises from 0.1 to 1% by weight, preferably from 0.1 to 0.8% by weight, particularly preferably from 0.1 to 0.5% by weight, of boron or phosphorus or a mixture of boron and phosphorus, in each case in oxidic form.

In a further preferred embodiment of the process of the invention, the catalyst in process step d) further comprises from 0.01 to 0.5% by weight, preferably from 0.1 to 0.4% by weight, of sulfur in oxidic form, with the proviso that the ratio of sulfur to nickel is in the range from 0.01 to 0.1 mol/mol.

In a further preferred embodiment of the process of the invention, the C4 mixture used for the selective hydrogenation in process step a) additionally comprises n-butane, isobutane or isobutene or mixtures thereof.

The isomerization is carried out at a temperature at which relocation of the double bond is ensured but skeletal isomerizations and oligomerizations are very largely avoided. The reaction temperature is therefore generally from 20 to 200° C., preferably from 20 to 120° C., particularly preferably from 30 to 110° C. The pressure is set so that the olefin stream is in liquid form. It is generally from 1 to 200 bar, preferably from 1 to 100 bar, particularly preferably from 4 to 30 bar.

In a further preferred embodiment of the process of the invention, the ratio of 2-butene to 1-butene at the outlet of the isomerization stage is in the range from 5 to 40, particularly preferably from 10 to 30.

The present invention is illustrated by the following examples.

### EXAMPLE 1

### Comparative Example 1

### [NiOₓ/Al₂O₃]

200 g of an Al₂O₃ support material (1.5 mm extrudates D10-10 from BASF AG) were treated with a solution comprising 108 g of Ni(NO₃)₂*6H₂O made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 9.0% by weight. The catalyst extrudates were comminuted and pressed through a sieve having an upper mesh opening of 1.0 mm and a lower mesh opening of 0.5 mm.

### EXAMPLE 2

### Comparative Example 2

### [NiₐS_{b}Oₓ/Al₂O₃]

200 g of an Al₂O₃ support material (1.5 mm extrudates D10-10 from BASF AG) were treated with a solution comprising 108 g of Ni(NO₃)₂*6H₂O and 6.4 g of H₂SO₄ (96% strength solution, calculated 100%) made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.8% by weight and an S content of 0.8% by weight, corresponding to an S:Ni ratio of 0.17 mol/mol. The catalyst extrudates were comminuted and pressed through a sieve having an upper mesh opening of 1.0 mm and a lower mesh opening of 0.5 mm.

### EXAMPLE 3

### Inventive Example 1

### [NiₐW_{b}Oₓ/Al₂O₃]

200 g of an Al₂O₃ support material (1.5 mm extrudates D10-10 from BASF AG) were treated with a solution comprising 108 g of Ni(NO₃)₂*6H₂O and 31 g of (NH₄)₆H₂W₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.3% by weight and a W content of 8.8% by weight. The catalyst extrudates were comminuted and pressed through a sieve having an upper mesh opening of 1.0 mm and a lower mesh opening of 0.5 mm.

### EXAMPLE 4

### Inventive Example 2

### [NiₐW_{b}Oₓ/Al₂O₃]

200 g of an Al₂O₃ support material (1.5 mm extrudates D10-10 from BASF AG) were treated with a solution comprising 108 g of Ni(NO₃)₂*6H₂O and 15.4 g of (NH₄)₆H₂W₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.3% by weight and a W content of 4.2% by weight. The catalyst extrudates were comminuted and pressed through a sieve having an upper mesh opening of 1.0 mm and a lower mesh opening of 0.5 mm.

### EXAMPLE 5

### Inventive Example 3

### [NiₐS_{b}W_{c}Oₓ/Al₂O₃]

200 g of an Al₂O₃ support material (1.5 mm extrudates D10-10 from BASF AG) were treated with a solution comprising 108 g of Ni(NO₃)₂*6H₂O, 3.2 g of H₂SO₄ (96% strength solution, calculated 100%) and 15.4 g of (NH₄)₆H₂W₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.7% by weight, an S content of 0.3% by weight (S:Ni=0.06) and a W content of 4.3% by weight. The catalyst extrudates were comminuted and pressed through a sieve having an upper mesh opening of 1.0 mm and a lower mesh opening of 0.5 mm.

### EXAMPLE 6

### Inventive Example 4

### [NiₐB_{b}W_{c}Oₓ/Al₂O₃]

200 g of an Al₂O₃ support material (1.5 mm extrudates D10-10 from BASF AG) were treated with a solution comprising 108 g of Ni(NO₃)₂*6H₂O, 4.1 g of H₃BO₃ and 15.4 g of (NH₄)₆H₂W₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.7% by weight, a B content of 0.3% by weight and a W content of 4.3% by weight. The catalyst extrudates were comminuted and pressed through a sieve having an upper mesh opening of 1.0 mm and a lower mesh opening of 0.5 mm.

### EXAMPLE 7

### Inventive Example 5

### [NiₐMo_{b}W_{c}Oₓ/Al₂O₃]

200 g of an Al₂O₃ support material (1.5 mm extrudates D10-10 from BASF AG) were treated with a solution comprising 108 g of Ni(NO₃)₂*6H₂O, 10.4 g of (NH₄)₆Mo₇O₂₄ and 15.4 g of (NH₄)₆H₂W₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.4% by weight, an Mo content of 2.1% by weight and a W content of 4.2% by weight. The catalyst extrudates were comminuted and pressed through a sieve having an upper mesh opening of 1.0 mm and a lower mesh opening of 0.5 mm.

### EXAMPLE 8

### Inventive Example 6

### [NiₐW_{c}Oₓ/Al₂O₃]

200 g of an Al₂O₃ support material (1.5 mm extrudates D10-D10 from BASF AG) were treated with a solution comprising 162.2 g of Ni(NO₃)₂*6H₂O and 30.7 g of (NH₄)₆H₂W₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 11.4% by weight and a W content of 7.6% by weight. The catalyst extrudates were comminuted and pressed through a sieve having an upper mesh opening of 1.0 mm and a lower mesh opening of 0.5 mm.

### EXAMPLE 9

### Inventive Example 7

### [NiₐW_{c}Oₓ/Al₂O₃]

200 g of an Al₂O₃ support material (1.5 mm extrudates D10-D10 from BASF AG) were treated with a solution comprising 108 g of Ni(NO₃)₂*6H₂O and 46.1 g of (NH₄)₆H₂W₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 7.6% by weight and a W content of 11.5% by weight. The catalyst extrudates were comminuted and pressed through a sieve having an upper mesh opening of 1.0 mm and a lower mesh opening of 0.5 mm.

### EXAMPLE 10

### Inventive Example 8

### [NiₐP_{b}Mo_{c}Oₓ/Al₂O₃]

200 g of an Al₂O₃ support material (1.5 mm extrudates D10-D10 from BASF AG) were treated with a solution comprising 108 g of Ni(NO₃)_{2*6}H₂O and 18.9 g of H₃PMo₁₂O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.7% by weight, a P content of 0.1% by weight and an Mo content of 3.6% by weight. The catalyst extrudates were comminuted and pressed through a sieve having an upper mesh opening of 1.0 mm and a lower mesh opening of 0.5 mm.

### EXAMPLE 11

### Inventive Example 9

### [NiₐV_{b}Mo_{c}Oₓ/Al₂O_{3]}

200 g of an Al₂O₃ support material (1.5 mm extrudates D10-10 from BASF AG) were treated with a solution comprising 108 g of Ni(NO₃)_{2*6}H₂O and 15.8 g of H₇PV₄Mo₈O₄₀ made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.7% by weight, a V content of 0.55% by weight, a P content of 0.09% by weight and an Mo content of 2.2% by weight. The catalyst extrudates were comminuted and pressed through a sieve having an upper mesh opening of 1.0 mm and a lower mesh opening of 0.5 mm.

### EXAMPLE 12

### Inventive Example 10

### [NiₐV_{b}W_{c}Oₓ/Al₂O₃]

14.8 g of oxalic acid were dissolved in 83 ml of H₂O and heated to 50° C. After addition of 5.6 g of V₂O₅, the solution was heated to 85° C. until a clear blue solution had been formed. After cooling to room temperature, 200 g of an Al₂O₃ support material (1.5 mm extrudates D10-10 from BASF AG) were treated with a solution comprising 108 g of Ni(NO₃)₂*6H₂O, 15.4 g of (NH₄)₆H₂W₁₂O₄₀ and the above vanadyl oxalate solution made up with H₂O to a volume of 160 ml in a rotating flask. After drying overnight at 120° C. in a drying oven, the dried catalyst was calcined in a rotary tube at 500° C. for 2 hours while passing 300 l of air/h through the tube. Elemental analysis indicated an Ni content of 8.7% by weight, a V content of 1.2% by weight and a W content of 4.3% by weight. The catalyst extrudates were comminuted and pressed through a sieve having an upper mesh opening of 1.0 mm and a lower mesh opening of 0.5 mm.

### EXAMPLE 13

### Catalyst Tests

### Comparative Catalysts 1 and 2 Vs. Catalysts According to the Invention 1 to 10

The experiments were carried out in a laboratory plant comprising eight tube reactors which had a length of 25 cm and an internal diameter of 1 cm and were to be operated in parallel and were installed in a convection oven in order to control the temperature. Each reactor was filled firstly with 5 ml of inert material and then with 15 g of catalyst in the form of 0.5 to 1 mm crushed material. After activation of the catalysts in a stream of N₂ (10 standard l/h per reactor, atmospheric pressure) at 250° C. for a period of 16 hours, the reactors were cooled to below 30° C. in a stream of N₂. After switching off the nitrogen supply, the reactors were pressurized with the olefin starting material to the reaction pressure of 20 bar. Finally, introduction of the feed by means of an HPLC pump was set to the desired value. To ensure satisfactory wetting of the catalyst and reproducible conditions (stirred tank characteristics), a centrifugal pump to circulate the feed was additionally operated at a ratio of about 20:1 (recycle:feed).

In selected experiments, the circulating pump was switched off to simulate plug flow characteristics in these examples.

The experiments were carried out using raffinate II having the composition 42% of 1-butene, 32% of 2-butenes, 2% of i-butene, 24% of butanes, <100 ppm of butadiene and one experiment was repeated using 1-dodecene (96% strength).

Analysis was carried out by means of on-line GC analysis (GC % by area).

The following tables 1 and 2 report the results obtained after a running time of 48 h:

for comparison, thermodynamic equilibrium data 2-butene:1-butene ratio 40° C.:30; 60° C.:23; 80° C.:18; 100° C.:16; 120° C.:14; 250° C.:8 (from D. R. Stull, E. F. Westrum, G. C. Sinke, The Chemical Thermodynamics of Organic Compounds, John Wiley & Sons, New York 1969 and table in U.S. Pat. No. 5,177,281, p. 7)

TABLE 1
Feed: raffinate II

| | | Temp. | WHSV | 1-Butene | 2-Butene | | Oligomers | |
|---|---|---|---|---|---|---|---|---|
| Catalyst | Circulation | ° C. | g/g*h | % | % | 2-Bu/1-Bu | % | Total |
| D10-10 | yes | 80 | 0.3 | 41 | 31 | 1 | 2 | 74 |
| Comparison 1 | yes | 80 | 0.3 | 37 | 36 | 1 | 1 | 74 |
| Comparison 2 | yes | 60 | 0.3 | 12 | 58 | 5 | 4 | 74 |
| | | 80 | 0.3 | 4 | 61 | 15 | 9 | 74 |
| Inventive 1 | yes | 60 | 0.3 | 6 | 65 | 11 | 2 | 73 |
| | yes | 80 | 0.3 | 4 | 66 | 17 | 3 | 73 |
| | no | 60 | 0.3 | 3 | 68 | 23 | 2 | 73 |
| | no | 60 | 0.7 | 5 | 67 | 13 | 2 | 74 |
| | no | 60 | 1.1 | 6 | 66 | 11 | 1 | 73 |
| | no | 45 | 0.3 | 6 | 66 | 11 | 1 | 73 |
| Inventive 2 | yes | 100 | 0.3 | 9 | 64 | 7 | 1 | 74 |
| | no | 100 | 0.3 | 7 | 65 | 9 | 2 | 74 |
| Inventive 3 | yes | 100 | 0.3 | 9 | 63 | 7 | 2 | 74 |
| Inventive 4 | yes | 80 | 0.3 | 9 | 63 | 7 | 2 | 74 |
| Inventive 5 | yes | 80 | 0.3 | 8 | 64 | 8 | 2 | 74 |
| Inventive 6 | yes | 80 | 0.3 | 9 | 63 | 7 | 2 | 74 |
| Inventive 7 | yes | 60 | 0.6 | 4 | 64 | 16 | 6 | 74 |
| | no | 60 | 2 | 4 | 68 | 17 | 2 | 74 |
| | no | 60 | 2.5 | 6 | 66 | 11 | 2 | 74 |
| | no | 40 | 0.6 | 4 | 68 | 17 | 2 | 74 |
| Inventive 8 | yes | 100 | 0.3 | 7 | 63 | 9 | 4 | 74 |
| Inventive 9 | yes | 100 | 0.3 | 12 | 61 | 5 | 1 | 74 |
| Inventive 10 | yes | 100 | 0.3 | 12 | 61 | 5 | 1 | 74 |

TABLE 2
Feed: 1-dodecene

| | Circu- | | | 1- | Internal | |
|---|---|---|---|---|---|---|
| Catalyst | lation | Temp. | WHSV | Dodecene | dodecenes | Oligomers |
| Inven- | yes | 80 | 0.3 | 38 | 57 | 2 |
| tive 1 | | | | | | |

### EXAMPLE 14

### Metathesis Reaction

### 14/1—Catalyst Production for the Metathesis Reaction:

Part A: 235.2 g of SiO₂ extrudates (BASF) having a diameter of 1.5 mm were impregnated to water uptake with an aqueous solution composed of 30.9 g of ammonium metatungstate and 635 g of water. After 15 minutes, the extrudates were predried at 80° C. and 50 mbar on a rotary evaporator, then dried overnight at 120° C. in a vacuum drying oven and finally calcined at 600° C. in a stream of N₂.

Part B: 400 g of Al₂O₃ extrudates (BASF) having a diameter of 1.5 mm were impregnated to water uptake with an aqueous solution of 368.9 g of magnesium nitrate hexahydrate and 8.1 g of sodium nitrate made up to 281 ml. The extrudates were dried overnight at 120° C. in a drying oven and finally calcined at 500° C. in a stream of N₂.

14/2—Use of the Catalyst from Example 14/1 in the Metathesis Reaction:

20 g of catalyst were installed as a mixture of 5 g of catalyst as per example 14/1—part A and 15 g of catalyst as per example 14/1—part B in the form of 1.5 mm extrudates in a tube reactor. The catalyst was activated by passing air over it at 600° C., made inert in a stream of N₂ while cooling to 530° C. and a raffinate stream was passed over it while simultaneously cooling to the reaction temperature. The feed comprised ethylene and raffinate 2H. As raffinate 2H, a mixture of about 85% by weight of linear butenes, about 2.5% by weight of isobutene and butanes (balance to 100% by weight) was used directly (experiment 1) or after enrichment with 2-butene (experiment 2). The reaction was carried out at 300° C. and 25 bar. The inlet and outlet compositions were determined by means of on-line GC. Table 3 below shows the conversions and mass selectivities for the metathesis for the two experiments having a different 2-butene/1-butene ratio in the raffinate. The mass selectivity here indicates the proportion by mass of propene in the product (propene plus olefins of C₅ and above). The higher 2-butene/1-butene ratio in experiment 2 corresponds to a feed as is supplied from the isomerization according to the invention.

TABLE 3

| | | | | 2-Butene/ | | |
|---|---|---|---|---|---|---|
| | Ethylene | | Ethylene/ | 1-butene | Butene | Propene |
| | standard | n-Butenes | n-butenes | (feed | conversion | selectivity |
| | l/h | g/h | molar | stream) | % | % |
| Experiment 1 | 62 | 100 | 1.56 | 0.8 | 44 | 89 |
| Experiment 2 | 62 | 100 | 1.55 | 5.5 | 55 | 94 |

It can be seen that a higher proportion of 2-butene in the feed results in more propylene being formed, which is shown by an increase in the butene conversion and the propene selectivity in experiment 2 in which the proportion of 2-butene is higher.

1-30. (canceled)

31. A process for isomerizing olefins in olefin-comprising hydrocarbon mixtures having from 4 to 20 carbon atoms at temperatures of from 20 to 200° C. and pressures of from 1 to 200 bar in the liquid phase in the presence of a heterogeneous catalyst, wherein a catalyst comprising from 1 to 20% by weight of nickel in oxidic form and from 1 to 20% by weight of at least one element of group VIB on an aluminum oxide support is used.

32. The process according to claim 31, wherein the catalyst further comprises from 0.1 to 10% by weight of one or more elements of group VB in oxidic form.

33. The process according to claim 31, wherein the catalyst further comprises from 0.1 to 1% by weight of boron or phosphorus or a mixture of boron and phosphorus, in each case in oxidic form.

34. The process according to claim 31, wherein the catalyst further comprises from 0.001 to 0.5% by weight of sulfur in oxidic form, with the proviso that the ratio of sulfur to nickel is in the range from 0.01 to 0.1 mol/mol.

35. The process according to claim 31, wherein gamma-, eta- or theta-aluminum oxide or a mixture thereof is used as support material.

36. The process according to claim 35, wherein gamma-aluminum oxide is used as support material.

37. The process according to claim 31, wherein the isomerization is combined with a selective hydrogenation.

38. The process according to claims 31, wherein the isomerization is combined with an olefin metathesis.

39. The process according to claim 31, wherein the isomerization is combined with an olefin alkylation.

40. The process according to claim 31, wherein the isomerization is combined with an olefin oligomerization.

41. A process for preparing propylene from n-butenes and ethylene by
a) selective hydrogenation of a C4 mixture comprising essentially a mixture of 1- and 2-butenes and butadiene;
b) isomerization of the 1-butene in the selectively hydrogenated C4 mixture at temperatures of from 20 to 200° C. and pressures of from 1 to 200 bar in the liquid phase in the presence of a heterogeneous catalyst;
c) metathesis of ethylene with 2-butene to form propylene in the selectively hydrogenated and isomerized C4 mixture in the presence of a metathesis catalyst, with at least equimolar amounts of ethylene based on the butenes comprised in the C4 mixture being used.

42. The process according to claim 41, wherein the isomerization in process step b) is carried out without addition of hydrogen.

43. The process according to claim 41, wherein a catalyst comprising from 1 to 20% by weight of nickel in oxidic form and from 1 to 20% by weight of at least one element of group VIB on an aluminum oxide support is used in process step b).

44. The process according to claim 43, wherein the catalyst further comprises from 0.1 to 10% by weight of one or more elements of group VB in oxidic form.

45. The process according to claim 43, wherein the catalyst further comprises from 0.1 to 1% by weight of boron or phosphorus or a mixture of boron and phosphorus, in each case in oxidic form.

46. The process according to claim 43, wherein the catalyst further comprises from 0.01 to 0.5% by weight of sulfur in oxidic form, with the proviso that the ratio of sulfur to nickel is in the range from 0.01 to 0.1 mol/mol.

47. The process according to claim 41, wherein the C4 mixture used for the selective hydrogenation in process step a) additionally comprises n-butane, isobutane or isobutene or mixtures thereof.

48. The process according to any of claims 41, wherein d) the product stream obtained from the metathesis c) is firstly separated by distillation into a low boiler fraction A comprising C2-C3-olefins and a high boiler fraction B comprising C4-C6-olefins and butanes;
e) the low boiler fraction A obtained from d) is subsequently separated by distillation into an ethylene-comprising fraction and a propylene-comprising fraction, with the ethylene-comprising fraction being recirculated to process step c) and the propylene-comprising fraction being discharged as product, and
f) the high boiler fraction B obtained from d) is combined with the selectively hydrogenated C4 mixture obtained in process step a) and reused in the isomerization b).

49. The process according to any of claims 41, wherein
d) the product stream obtained from the metathesis c) is firstly separated by distillation into a low boiler fraction A comprising C2-C3-olefins and a high boiler fraction B comprising C4-C6-olefins and butanes;
e) the low boiler fraction A obtained from d) is subsequently separated by distillation into an ethylene-comprising fraction and a propylene-comprising fraction, with the ethylene-comprising fraction being recirculated to process step c) and the propylene-comprising fraction being discharged as product, and
f) the high boiler fraction B obtained from d) is combined with the selectively hydrogenated and isomerized C4 mixture obtained in process step b) and reused in the metathesis c).

50. The process according to claim 41, wherein the isomerization occurs at temperatures of from 30 to 110° C. and pressures of from 4 to 30 bar.

51. The process according to claim 41, wherein the ratio of 2-butene to 1-butene at the outlet of the isomerization stage b) is in the range from 5 to 40.

52. The process according to claim 51, wherein the ratio of 2-butene to 1-butene at the outlet of the isomerization stage is in the range from 10 to 30.

53. A process for preparing propylene from n-butenes and ethylene by
a) selective hydrogenation of a C4 mixture comprising essentially a mixture of 1- and 2-butenes and butadiene;
b) metathesis of ethylene with 2-butene to form propylene in the selectively hydrogenated C4 mixture in the presence of a metathesis catalyst, with at least equimolar amounts of ethylene based on the butenes comprised in the C4 mixture being used;
c) separation of propylene from the reaction mixture;
d) isomerization of the 1-butene in the C4 reaction mixture which has been freed of propylene at temperatures of from 20 to 200° C. and pressures of from 1 to 200 bar in the liquid phase in the presence of a heterogeneous catalyst and
e) combination of the streams from stages a) and d) and reuse in the metathesis stage b),
wherein a catalyst comprising from 1 to 20% by weight of nickel in oxidic form and from 1 to 20% by weight of at least one element of group VIB on an aluminum oxide support is used in process step d).

54. The process according to claim 53, wherein the catalyst in process step d) further comprises from 0.1 to 10% by weight of one or more elements of group VB in oxidic form.

55. The process according to claim 53, wherein the catalyst in process step d) further comprises from 0.1 to 1% by weight of boron or phosphorus or a mixture of boron and phosphorus, in each case in oxidic form.

56. The process according to claim 53, wherein the catalyst in process step d) further comprises from 0.01 to 0.5% by weight of sulfur in oxidic form, with the proviso that the ratio of sulfur to nickel is in the range from 0.01 to 0.1 mol/mol.

57. The process according to claim 53, wherein the C4 mixture used for the selective hydrogenation in process step a) additionally comprises n-butane, isobutane or isobutene or mixtures thereof.

58. The process according to claim 53, wherein the isomerization in process step d) occurs at temperatures of from 30 to 110° C. and pressures of from 4 to 30 bar.

59. The process according to claim 53, wherein the ratio of 2-butene to 1-butene at the outlet of the isomerization stage is in the range from 5 to 40.

60. The process according to claim 59, wherein the ratio of 2-butene to 1-butene at the outlet of the isomerization stage is in the range from 10 to 30.
